(19) United States
(12) Patent Application Publication
(10) Pub. No.: US 2019/0343083 A1
(43) Pub. Date: Nov. 14, 2019

(54) MEASURING INSTRUMENT AND BIOLOGICAL INFORMATION MEASURING SYSTEM

(71) Applicant: Sharp Kabushiki Kaisha, Sakai City, Osaka (JP)

(72) Inventors: Azusa NAKANO, Sakai City (JP); Hiroshi SAKAYA, Sakai City (JP); Tetsuya HAYASHI, Sakai City (JP); Keiji TAKEDA, Sakai City (JP)

(21) Appl. No.: 16/479,951

(22) PCT Filed: Feb. 5, 2018
(86) PCT No.: PCT/JP2018/003852

§ 371 Date: Jul. 23, 2019

(30) Foreign Application Priority Data
Feb. 6, 2017 (JP) 2017-019874

(51) Int. Cl. A01K 27/00; A01K 29/00

(52) U.S. Cl. CPC A01K 27/002; A01K 29/005; A01K 27/006

(57) ABSTRACT

An easy-to-fit measuring instrument is provided. A measuring instrument (10) includes: a main wearable unit (1) to be fitted either to a neck of an animal on which measurement is to be made or close to the neck; and an electrode belt (5) including an electrode (6) to be fitted, for measurement of biological information, in such a manner that the electrode is in contact with at least either one of left and right axillae of the animal, wherein the electrode belt has a first end that is connected to the main wearable unit and a second end that is to be connected to the main wearable unit in a location that is closer to a back of the animal than the first end is close to the back of the animal.

### TECHNICAL FIELD

The present disclosure relates to measuring instruments to be worn by a living body, in particular, a haired animal and relates also to biological information measuring systems that include such a measuring instrument.

### BACKGROUND ART

It is widely recognized that everyday health management plays an important role in the prevention and treatment of lifestyle diseases. Awareness is growing that everyday health management is just as important in companion animals as in humans (owners of animals) There is an increasing need for owners to readily measure biological information of their companion animals.

An exemplary technique of measuring biological information of a companion animal is described in Patent Literature 1 listed below. A measuring instrument disclosed in Patent Literature 1 includes a fixture for holding electrodes of electrode members pressed to armpits or other body parts.

### CITATION LIST

### Patent Literature

Patent Literature 1: Japanese Unexamined Patent Application Publication, Tokukai, No. 2005-27661A (Publication Date: Feb. 3, 2005)

### SUMMARY OF INVENTION

### Technical Problem

Animals are not always obedient while owners are fitting a measuring instrument to them. The measuring instrument disclosed in Patent Literature 1 has room for improvement in ease of fitting.

The present disclosure, in an aspect thereof, has an object to provide, for example, an easy-to-fit measuring instrument.

### Solution to Problem

The present disclosure, in an aspect thereof, is directed to a measuring instrument including: a main wearable unit to be fitted either to a neck of an animal on which measurement is to be made or close to the neck; and an electrode belt including an electrode to be fitted, for measurement of biological information, in such a manner that the electrode is in contact with at least either one of left and right axillae of the animal, wherein the electrode belt has a first end that is connected to the main wearable unit and a second end that is to be connected to the main wearable unit in a location that is closer to a back of the animal than the first end is close to the back of the animal.

### Advantageous Effects of Invention

The present disclosure, in an aspect thereof, can provide an easy-to-fit measuring instrument.

### BRIEF DESCRIPTION OF DRAWINGS

FIG. 1 is an illustration of an exemplary appearance of a measuring instrument in accordance with Embodiment 1.

FIG. 2 is a set of illustrations showing how the measuring instrument in accordance with Embodiment 1 is fitted to a dog, (a) of FIG. 2 representing halfway through the fitting process and (b) of FIG. 2 representing the measuring instrument having been completely fitted.

Portion (a) of FIG. 3 is an illustration of a structure of an electrode belt in accordance with Embodiment 2. Portion (b) of FIG. 3 is an illustration of an exemplary structure of a length adjusting section. Portion (c) of FIG. 3 is an illustration of another exemplary structure of the length adjusting section. Portion (d) of FIG. 3 is an illustration of a further exemplary structure of the length adjusting section. Portion (e) of FIG. 3 is an illustration of an exemplary use of an extension belt as a length adjusting section.

FIG. 4 is an illustration of an exemplary appearance of a measuring instrument in accordance with Embodiment 3.

FIG. 5 is a development of the measuring instrument shown in FIG. 4.

FIG. 6 is a set of illustrations of the measuring instrument in accordance with Embodiment 3 being fitted to a dog, (a) of FIG. 6 showing the dog as viewed from a side of the dog and (b) of FIG. 6 showing the dog as viewed from above the dog.

FIG. 7 is a functional block diagram of a configuration of an electrocardiograph system in accordance with Embodiment 4.

FIG. 8 is a diagram of an exemplary hardware configuration of an electrocardiograph included in the electrocardiograph system in accordance with Embodiment 4.

FIG. 9 is a flow chart representing an exemplary flow of a proper fitting check process in an electrocardiograph included in the electrocardiograph system in accordance with Embodiment 4.

FIG. 10 is a set of diagrams representing exemplary electrocardiographic waveforms, (a) of FIG. 10 representing a waveform obtained when electrodes are properly fitted and (b) of FIG. 10 representing a waveform obtained when electrodes are not properly fitted.

### DESCRIPTION OF EMBODIMENTS

The following will describe examples where the animal on which measurement is carried out is a dog. The animal on which measurement is carried out is not necessarily a dog and may be, for example, (i) a companion animal such as a cat, a rabbit, a ferret, a monkey, or a hamster, (ii) a farm animal such as a horse, a cow, a pig, a sheep, or a goat, or (iii) a zoo animal such as a tiger or a lion.

### Embodiment 1

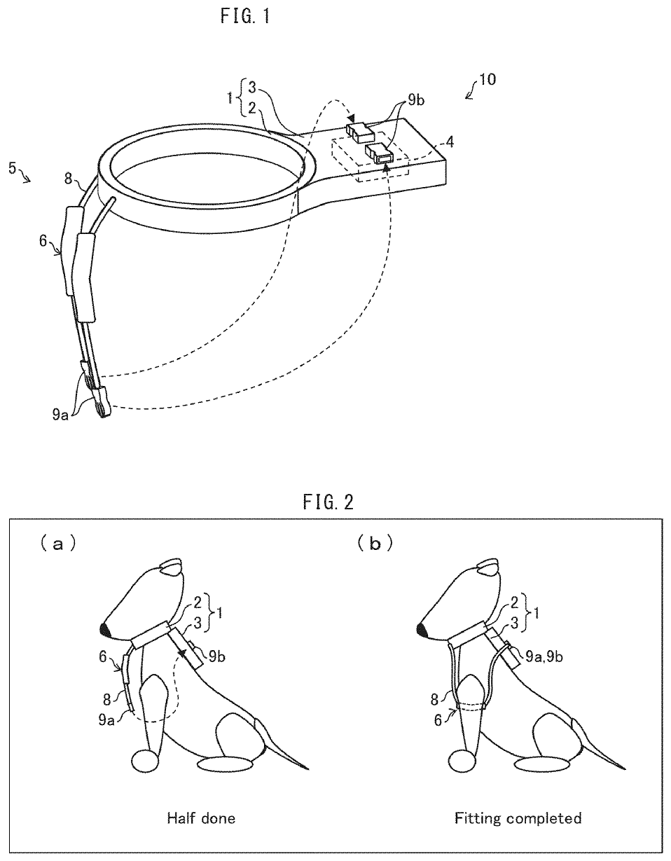

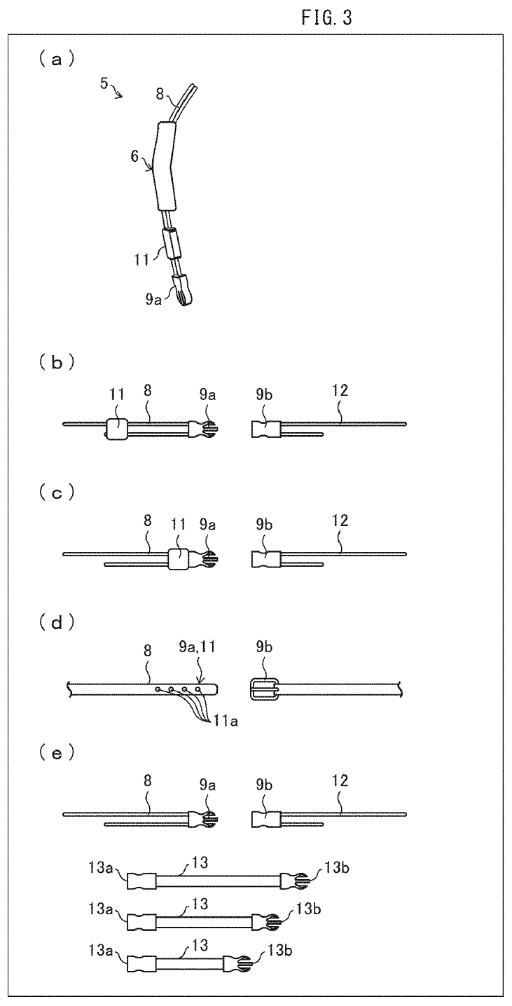

A description is now given of an embodiment with reference to FIGS. 1 to 2.

FIG. 1 is an illustration of an exemplary appearance of a measuring instrument 10 in accordance with the present embodiment. The measuring instrument 10 is a measuring instrument that, for example, measures cardiac electrical activity on a dog. The measuring instrument 10, however, does not necessarily measure cardiac electrical activity and may alternatively measure, for example, body temperature, pulse wave, perspiration rate, heart rate, body fat percentage, or other biological information. The measuring instrument 10 includes a main wearable unit 1 and electrode belts 5 as shown in FIG. 1.

The main wearable unit 1 is a member fitted to or near the neck of a dog and includes a collar section 2 and a casing 3. The collar section 2 is an annular member that serves as a collar worn by the dog. This collar section 2 needs only to be annular when worn by the dog.

Accordingly, an apparently annular collar section 2 may be provided by fitting a plurality of straps so as to cross around the neck of the dog or by connecting a plurality of straps. The collar section 2 is not necessarily made of a particular substance and may be made of any substance including natural leather, synthetic leather, resin, natural fiber, and chemical fiber.

The casing 3 sits on the back of the dog when the collar section 2 is fitted to the dog. The casing 3 contains therein an electrocardiograph 4 (analysis system) that measures cardiac electrical activity on the dog. The electrocardiograph 4 will be described later in detail.

The electrode belts 5 include electrodes 6 for measuring cardiac electrical activity as biological information and are fitted such that the electrodes 6 come into contact with the left and right axillae of the dog. The electrode belts 5 are not necessarily fitted under both the left and right axillae of the dog. One of the electrode belts 5 may be sufficiently fitted under either the left or right axilla. Each electrode belt 5 includes one of the electrodes 6, a belt 8, and a coupling section 9a.

Each electrode 6 is positioned under an axilla of the dog when the measuring instrument 10 is fitted. In an axilla, the dog's body has a surface area with relatively sparse fur. Therefore, by attaching the electrodes 6 in contact with the axillae, cardiac electrical activity can be measured without having to shave the fur, apply electrically conductive gel, or perform another similarly troublesome process. The axillae are unlikely to come into contact with, for example, the floor or wall. For this reason, the electrodes 6 will remain in place, which contributes to successful normal measurement. FIG. 1 shows that two electrodes 6 are provided for one measuring instrument 10. However, any number of electrodes 6 may be provided in accordance with the physical properties that the measuring instrument 10 is designed to measure.

Each belt 8 supports one of the electrodes 6 to attach the electrode 6 in contact with the dog's axilla with a prescribed or greater force. The belt 8 has one of the ends thereof being connected to the collar section 2 of the main wearable unit 1. The belt 8 is partly or entirely made of, for example, rubber or another elastic substance. For instance, the belt 8 may be provided by connecting a rubber belt with a poorly stretchable or non-stretchable belt. In this structure, the coupling section 9a may be provided on the poorly stretchable or non-stretchable belt.

The belts 8 each include an elastic portion. The elasticity of the belts 8 maintains the electrodes 6 in contact with the dog's axillae even when the dog wearing the measuring instrument 10 changes its posture. The belts 8 may be provided by a string member with moderate elasticity.

Each coupling section 9a is coupled to a coupling section 9b in a detachable manner to connect an end of the electrode belt 5 to the main wearable unit 1 in a detachable manner. The coupling section 9a is provided on an end (second end) of the belt 8 opposite from the other end thereof (first end) connected to the collar section 2. The coupling sections 9b may be provided on the casing 3, for example, as shown in FIG. 1. Alternatively, the coupling sections 9b may be provided on the collar section 2.

The coupling sections 9b, in the present embodiment, are provided on a part of the collar section 2 that corresponds to the dog's back. Therefore, the second ends of the electrode belts 5 are coupled to the main wearable unit 1 on the dog's back. Depending on the dog's body size, the electrodes 6 may readily and properly come into contact with the axillae if the coupling sections 9b are provided on the casing 3 (in other words, on the dog's back) rather than on the collar section 2 (e.g., exactly above the dog's axillae). The second ends of the electrode belts 5 need only be capable of being coupled to the main wearable unit 1 at positions that are at least closer to the dog's back than the first ends are close to the dog's back.

FIG. 2 is a set of illustrations showing how the measuring instrument 10 is fitted to a dog. To fit the measuring instrument 10 to a dog, the collar section 2 is first fitted around the neck of the dog with the coupling sections 9a and 9b being uncoupled as shown in (a) of FIG. 2. Thereafter, the electrode belts 5 are passed under the dog's armpits and pulled up. The coupling sections 9a are then coupled to the coupling sections 9b, which completes the fitting of the measuring instrument 10 to the dog as shown in (b) of FIG. 2.

Many dogs have hair on their skin. The user needs to either shave the hair or choose sites where the dog has relatively thin body hair as measurement points, to measure cardiac electrical activity on the dog's skin. It is difficult to make accurate cardiac electrical activity measurement at sites with thick body hair because the body hair acts as a noise source or an insulator. Although the user can improve contact between electrodes and the body surface, for example, by using electrically conductive gel, the gel loses its electrical conductivity once it dries and may smear the body hair.

To take measurement over an extended period of time without shaving, a preferred method should allow the electrodes to be installed at sites where the dog has relatively thin body hair, remain in place for an extended period of time, and if displaced, return to the original positions. Another requirement is a tool that enables anyone to repeatedly implement this method.

The collar section 2 is a collar as described earlier and formed not to drop below the dog's shoulders. Therefore, when the measuring instrument 10 is fitted to the dog, the electrodes 6 come into contact with the dog's axillae with a desired pressure due to the tension in the electrode belts 5. The axillae have relatively thin body hair and provide preferred measurement points for measurement of cardiac electrical activity and other biological information.

To fit the measuring instrument 10, the free ends (second ends) of the electrode belts 5 are passed under the dog's axillae from the front toward the back of the dog and coupled to the main wearable unit 1 on the dog's back as shown in FIG. 2. If the measuring instrument 10 was structured such that the electrode belts 5 be coupled to the main wearable unit 1 on the front of the dog, and the user fails to smoothly and swiftly fit the measuring instrument 10, the dog might, for example, bite the hands of the user trying to fit the measuring instrument 10, depending on the dog's personality. Since the measuring instrument 10 is structured such that the electrode belts 5 can be connected to the main wearable unit 1 on the dog's back, the user will less likely face such a risk.

Additionally, since the coupling sections 9a are passed under the dog's axillae before being coupled to the coupling sections 9b, the measuring instrument 10 can be more easily fitted to the dog than in a structure where both ends of the electrode belts 5 are fixed to the collar section 2 in advance.

The fitting of the measuring instrument 10 constricts only the motion of the dog's axillae. The structure constricts the motion of a minimum number of body parts of the dog. Meanwhile, the user, when fitting the measuring instrument 10 to the dog, can readily install the electrodes 6 in place by simply placing the collar section 2 around the neck of the dog and then coupling the coupling sections 9a to the coupling sections 9b. Therefore, the user can install the electrodes 6 in contact with proper sites to make measurement with high reproducibility even if the user (e.g., the dog's owner) is not familiar with the measurement of cardiac electrical activity.

### Embodiment 2

The following will describe another embodiment with reference to FIG. 3. For convenience of description, members of the present embodiment that have the same function as members of the previous embodiment are indicated by the same reference numerals, and description thereof is omitted.

Portion (a) of FIG. 3 is an illustration of a structure of an electrode belt 5 in accordance with the present embodiment. As shown in (a) of FIG. 3, each electrode belt 5 of the present embodiment includes a length adjusting section 11 as well as those members shown in FIG. 1. The length adjusting sections 11 have (i) a function of adjusting the lengths of the belts 8 and (ii) a function of retaining the adjusted lengths (length retaining function). The provision of the length adjusting sections 11 on the electrode belts 5 enables the user of the measuring instrument 10 to have the electrodes 6 in contact with the dog's axillae with a proper pressure.

Portion (b) of FIG. 3 is an illustration of an exemplary structure of the length adjusting section 11. In the example shown in (b) of FIG. 3, the length adjusting section 11 is provided on the electrode belt 5 as a member that is separate from the coupling section 9a. Examples of such a length adjusting section 11 include adjusters and pin buckles. Examples of the coupling sections 9a and 9b include buckles, hooks, press studs, and buttons.

Portion (c) of FIG. 3 is an illustration of another exemplary structure of the length adjusting section 11. In the example shown in (c) of FIG. 3, the length adjusting section 11 is formed integrally with the coupling section 9a on the belt 8, in which case the length adjusting section 11 and the coupling section 9a may form, for example, an adjustable buckle.

Portion (d) of FIG. 3 is an illustration of a further exemplary structure of the length adjusting section 11. In the example shown in (d) of FIG. 3, the coupling sections 9a and 9b and the length adjusting section 11 provide a pin buckle. Specifically, the belt 8 is provided with a plurality of holes 11a that serves as the coupling section 9a and the length adjusting section 11. A pin, as the coupling section 9b, is inserted into one of the holes 11a to couple the coupling sections 9a and 9b together and also adjust the length of the electrode belt 5.

The length adjusting section 11 is provided on the belt 8 in all the examples shown in (b) to (d) of FIG. 3. The length adjusting section 11 may however be provided on a belt 12 on which the coupling section 9b is provided. The length adjusting section 11 may alternatively be provided on both the belt 8 and the belt 12.

If the belt 8 is formed entirely of rubber, it may be difficult to adjust the length of the electrode belt 5 using the length adjusting section 11. In addition, in the example shown in (d) of FIG. 3, the hole 11a may be expanded under tension when a pin as the coupling section 9b is inserted thereinto, which may give a different length than an intended length. For these reasons, the belt 8 may be a rubber belt attached to a poorly stretchable or non-stretchable belt, and the length adjusting section 11 may be disposed on the poorly stretchable or non-stretchable belt.

Portion (e) of FIG. 3 is an illustration of an exemplary use of an extension belt 13 as the length adjusting section 11. The extension belt 13 has on respective ends thereof a coupling section 13a that can be coupled to the coupling section 9a and a coupling section 13b that can be coupled to the coupling section 9b. The electrode belt 5 can be extended by coupling the belt 8 to the belt 12 via the extension belt 13. The length of the electrode belt 5 becomes adjustable so that the electrode 6 can be brought into contact with the dog's axilla under proper pressure, by preparing extension belts 13 of different lengths (three different lengths in (e) of FIG. 3) and selecting one of the extension belts 13 that has an optimal length in view of the body size of the dog to which the measuring instrument 10 is to be fitted.

If the length adjusting section 11 had no length retaining function, the user would need to adjust the length of the electrode belt 5 every time the measuring instrument 10 is fitted. The user would have to repeatedly perform a complex task of fitting the measuring instrument 10 on the same dog. Adjusting the length of the electrode belt 5 becomes troublesome especially when the measuring instrument 10 is already fitted to the dog, because tension is being produced in the electrode belt 5 to retain the electrode 6 in contact with the dog's axilla.

If the measuring instrument 10 had no coupling sections 9a and 9b, that is, if the electrode belt 5 had both ends thereof being fixed, the collar section 2 would need to be placed around the dog's neck, and the dog's forelimb would need to be passed through the annular section formed by the collar section 2 and the electrode belt 5. Therefore, it would be troublesome to fit the measuring instrument 10.

Additionally, it would be difficult to pass the dog's forelimb through the annular section if the electrode belt 5 had such a length that the measuring instrument 10 is being properly fitted to the dog. Therefore, to fit the measuring instrument 10, the electrode belt 5 may need to be temporarily extended or separated from the main wearable unit 1 at an end of the electrode belt 5. If the structure required that the electrode belt 5 be extended, it would become difficult to reproduce the length (tension) of the electrode belt 5 that is suited to the measurement of cardiac electrical activity every time the measuring instrument 10 is fitted.

The measuring instrument 10, once fitted to the dog with the length of the electrode belt 5 being properly adjusted using the length adjusting sections 11, allows anyone to readily reproduce in the electrode belt 5 the tension that is suited to the measurement of cardiac electrical activity of the dog in the second and subsequent fittings, by simply coupling the coupling sections 9a and 9b together.

More specifically, in the examples shown in (a), (b), and (c) of FIG. 3, once the length of the electrode belt 5 is adjusted using the length adjusting section 11, tension can be readily reproduced in the electrode belt 5 when the measuring instrument 10 is fitted to the dog for the second and subsequent times, by simply coupling the coupling sections 9a and 9b together. In the example shown in (d) of FIG. 3, when the measuring instrument 10 is fitted for the first time, the user checks which of the holes 11a (coupling section 9a) in the length adjusting section 11 should be selected to produce the most proper tension in the electrode belt 5. In the second and subsequent fittings of the measuring instrument 10, the user can readily reproduce proper tension in the electrode belt 5 by inserting a pin on the coupling section 9b into one of the holes 11a that produces the most proper tension. Meanwhile, in the example shown in (e) of FIG. 3, when the measuring instrument 10 is fitted for the first time, the user checks which of the extension belts 13 has a length that produces the most proper tension in the electrode belt 5. In the second and subsequent fittings of the measuring instrument 10, the user can readily reproduce proper tension in the electrode belt 5 by selectively using one of the extension belts 13 that has the proper length.

### Embodiment 3

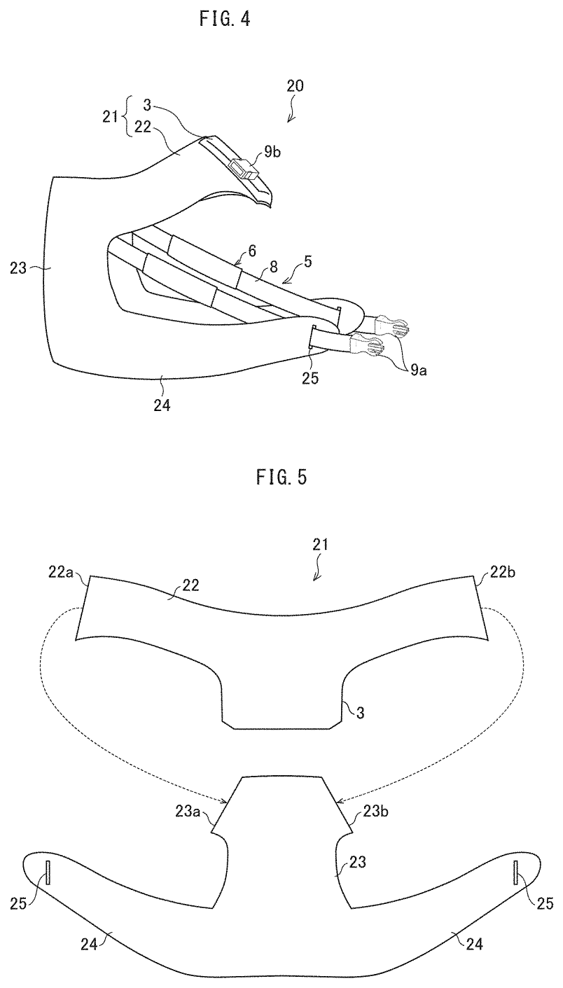

The following will describe another embodiment with reference to FIGS. 4 to 6. For convenience of description, members of the present embodiment that have the same function as members of a previous embodiment are indicated by the same reference numerals, and description thereof is omitted.

FIG. 4 is an illustration of an exemplary appearance of a measuring instrument 20 in accordance with the present embodiment. FIG. 5 is a development of the measuring instrument 20 shown in FIG. 4. Note that the electrode belts 5 are omitted from FIG. 5. Referring to FIGS. 4 and 5, the measuring instrument 20 includes a main wearable unit 21, electrode belts 5, a chest pad section 23, and axilla straps 24.

The main wearable unit 21 differs from the main wearable unit 1 in that the main wearable unit 21 includes a collar section 22 in place of the collar section 2. The collar section 22 is shaped like a strap with a greater width than the collar section 2, to cover the neck of a dog. The main wearable unit 21 may be made of any material including a cloth made of woven fabric of chemical or natural fibers. Specifically, the collar section 22 includes forelimb sections 22a, 22b shaped so as to cover around the neck of the dog. An annular member is formed by connecting the forelimb sections 22a, 22b to connecting sections 23a, 23b of the chest pad section 23.

At least the cloth that provides the collar section 22 (22a, 22b) is preferably non-stretchable or poorly stretchable so that the collar section 22 does not attenuate tension in the electrode belt 5. The cloth that provides the chest pad section 23 and the axilla straps 24 may be stretchable. The cloth that provides the collar section 22 (22a, 22b) and the chest pad section 23 may be quilting or a like fabric with cushioning properties.

The chest pad section 23 is connected to the main wearable unit 21 and positioned on the chest of the dog when the measuring instrument 20 is fitted to the dog. The chest pad section 23 includes, proximate to the shoulders of the dog when the measuring instrument 20 is fitted to the dog, the connecting sections 23a, 23b that are connected respectively to the forelimb sections 22a, 22b. The forelimb sections 22a, 22b may be connected to the connecting sections 23a, 23b in a detachable manner using, for example, press studs, fasteners, or buttons. One of the connecting sections 23a and 23b may be connected in an undetachable manner by, for example, sewing.

The axilla straps 24 are connected to the chest pad section 23 and positioned under the axillae (armpits) of the dog when the measuring instrument 20 is fitted to the dog. The axilla straps 24 are connected to the chest pad section 23 and extended toward the respective left and right axillae of the dog. Each axilla strap 24 has an insertion slot 25 through which the electrode belt 5 is passed. The chest pad section 23 and the axilla straps 24 may be made of, for example, cloth of the same material as the cloth for the collar section 22.

The measuring instrument 20, having the insertion slots 25 in the axilla straps 24, allows the user to pull up the axilla straps 24 toward the back of the dog by simply connecting the electrode belts 5 to the coupling sections 9b. Alternatively, the axilla straps 24 and the casing 3 may be structured such that the axilla straps 24 can be attached to the casing 3 or the periphery thereof, by providing both the axilla straps 24 themselves and the casing 3 or the periphery thereof with a hook and loop fastener or a hook. This structure enables the axilla straps 24 to be pulled up toward the back of the dog without having to provide the axilla straps 24 with the insertion slots 25. To do so, the user, for example, first couples the coupling sections 9a and 9b together and subsequently connects the unattached ends of the axilla straps 24 to the casing 3 or the periphery thereof, to fit the measuring instrument 10 to the dog.

In the measuring instrument 10, tension in the electrode belts 5 acts on the collar section 2 alone, thereby possibly placing a heavy load on the parts of the dog's neck that come into contact with the collar section 2. In contrast, in the measuring instrument 20, tension in the electrode belts 5 partly acts on the chest pad section 23 and is hence distributed. In addition, since the collar section 22 is formed with a greater width than the collar section 2 as described above, the collar section 22 mitigates the tension-caused pressure on the neck of the dog when compared with the collar section 2 in the measuring instrument 10, thereby reducing the load on the parts of the dog's neck that come in contact with the collar.

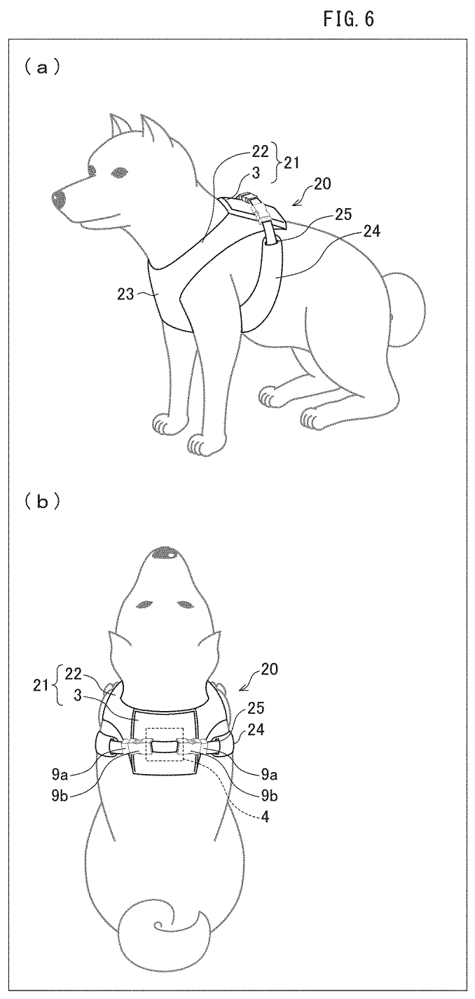

FIG. 6 is a set of illustrations of the measuring instrument 20 being fitted to a dog, (a) of FIG. 6 showing the dog as viewed from a side of the dog and (b) of FIG. 6 showing the dog as viewed from above the dog. As shown in (a) and (b) of FIG. 6, the measuring instrument 20 includes the collar section 22, the chest pad section 23, and the axilla straps 24, all of which are made of cloth. The measuring instrument 20 therefore has an appearance like animal clothes and still enables anyone to properly fit the electrodes 6. The appearance of the measuring instrument 20 gives good impressions to the dog's owner and other people around the dog when compared with an instrument that has such an appearance that people immediately know that it is an instrument when they see it.

### Embodiment 4

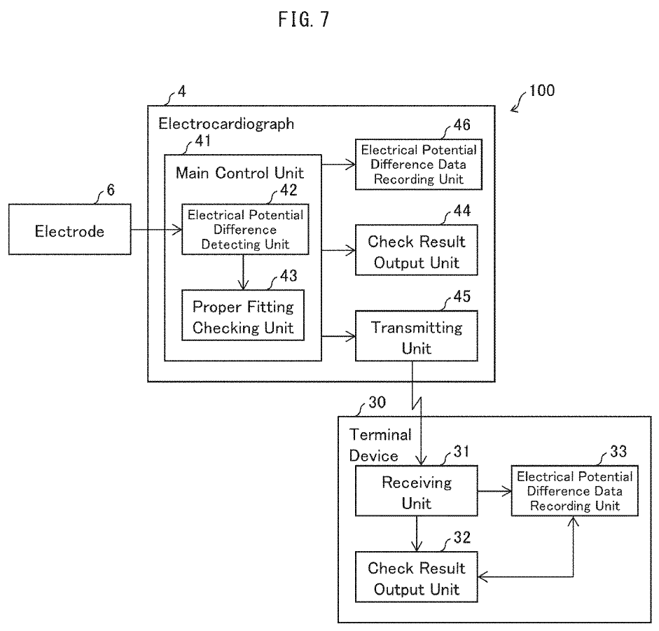

The following will describe another embodiment with reference to FIGS. 7 to 10. For convenience of description, members of the present embodiment that have the same function as members of a previous embodiment are indicated by the same reference numerals, and description thereof is omitted.

FIG. 7 is a functional block diagram of a configuration of an electrocardiograph system 100 (biological information measuring system). Referring to FIG. 7, the electrocardiograph system 100 includes electrodes 6 in the electrode belts 5 of the measuring instrument 10, an electrocardiograph 4 in the casing 3, and a terminal device 30 (analysis system). In other words, the electrocardiograph system 100 includes the measuring instrument 10, the electrocardiograph 4, and the terminal device 30. The electrocardiograph system 100 may include the measuring instrument 20 in place of the measuring instrument 10.

The electrocardiograph 4 includes a main control unit 41, a check result output unit 44, a transmitting unit 45, and an electrical potential difference data recording unit 46. The main control unit 41 is a functional block controlling the overall functionality of the electrocardiograph 4 and in particular includes an electrical potential difference detecting unit 42 and a proper fitting checking unit 43 (checking unit).

Cardiac electrical activity typically refers to minute electrical currents produced in a living body by, for example, heartbeats. Cardiac electrical activity is measured, for example, by placing a plurality of electrodes in locations across the heart and detecting electrical potential differences between the electrodes. The present disclosure describes examples where cardiac electrical activity is measured by measuring electrical potential differences between the electrodes 6 placed on the left and right forelegs. More particularly, the electrical potential difference detecting unit 42 detects differences between a first electrical potential difference detected by the electrode 6 placed under the axilla of the left foreleg and a second electrical potential difference detected by the electrode 6 placed under the axilla of the right foreleg (hereinafter, simply referred to as “electrical potential differences”) (biological information). The first and second electrical potential differences are a difference between the electric potential detected by the respective electrodes 6 and the reference potential. The main control unit 41 records the electrical potential difference data acquired by the electrical potential difference detecting unit 42 in the electrical potential difference data recording unit 46 which is a recording medium. The electrical potential difference data recording unit 46 is not necessarily included in the electrocardiograph 4 and may be provided in an external device. In the latter case, the main control unit 41 is, for example, connected to the external device via a wireless link to record the electrical potential difference data in the electrical potential difference data recording unit 46.

The proper fitting checking unit 43 analyzes the electrical potential differences to check whether or not the electrodes 6 are fitted properly to the living body on which a measurement is to be made (specifically, whether or not the measuring instrument 10 is fitted properly). Specifically, the proper fitting checking unit 43 calculates a difference between a maximum value and an average value of the electrical potential differences obtained during a prescribed period of time and if the difference between the maximum and average values is greater than a prescribed threshold value, determines that the electrodes 6 are properly fitted. On the other hand, if the difference is less than or equal to the prescribed threshold value, the proper fitting checking unit 43 determines that the electrodes 6 are not properly fitted. The main control unit 41 may record the results of the check performed by the proper fitting checking unit 43 in the electrical potential difference data recording unit 46 in association with the electrical potential difference data.

The check result output unit 44 outputs the results of the check performed by the proper fitting checking unit 43 and may be any device that alerts the user to the results of the check, including a speaker, a display device, or a light-emitting device such as an LED (light-emitting diode).

The transmitting unit 45 is a communications device that transmits the results of the check performed by the proper fitting checking unit 43 to the terminal device 30. The electrocardiograph 4 may communicate with the terminal device 30 through a wireless or wired link.

The terminal device 30 alerts the user located away from the electrocardiograph 4 to the results of the check performed by the proper fitting checking unit 43 and is, for example, a smartphone or like mobile terminal. The terminal device 30 includes a receiving unit 31 that receives the results of the check performed by the proper fitting checking unit 43 from the transmitting unit 45 and a check result output unit 32 that alerts the user to the results of the check. The check result output unit 32 is similar to the check result output unit 44.

The proper fitting checking unit 43 may be provided in the terminal device 30, in which case the transmitting unit 45 transmits the electrical potential difference data outputted from the electrical potential difference detecting unit 42 to the receiving unit 31 in the terminal device 30. Under the same condition, the terminal device 30 may further include an electrical potential difference data recording unit 33 that is a recording medium similar to the electrical potential difference data recording unit 46, to record the received electrical potential difference data and the results of the check performed by the proper fitting checking unit 43 included in the terminal device 30 in the electrical potential difference data recording unit 33 by associating these data and results.

The electrocardiograph system 100 may include only either the check result output unit 44 or the check result output unit 32. If the check result output unit 32 is not provided in the terminal device 30, and the check result output unit 44 is provided in the electrocardiograph 4, the electrocardiograph system 100 does not necessarily include the terminal device 30.

The electrical potential difference data recording unit 46 may record either all or part of the electrical potential difference data acquired by the electrical potential difference detecting unit 42. As a latter example, the electrical potential difference data recording unit 46 may record the electrical potential difference data acquired by the electrical potential difference detecting unit 42 only when the proper fitting checking unit 43 has determined that the electrodes 6 are properly fitted.

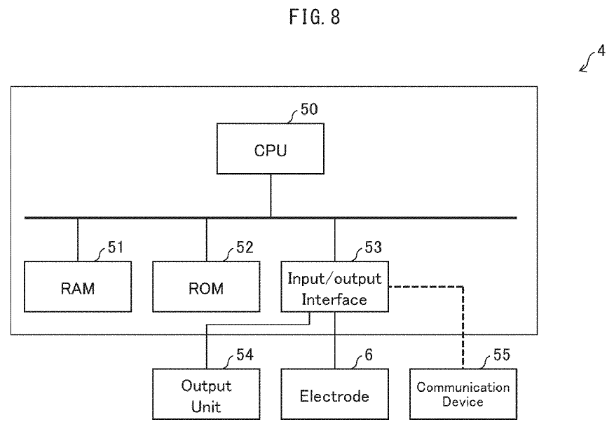

FIG. 8 is a diagram of an exemplary hardware configuration of the electrocardiograph 4. Referring to FIG. 8, the electrocardiograph 4 includes: a CPU 50 that executes instructions from programs or software by which the functions of the main control unit 41 are implemented; a ROM (read-only memory) 52 containing the programs and various data in a format readable by the CPU 50; a RAM (random access memory) 51 into which the programs are loaded; and an input/output interface 53 via which data is exchanged to and from an external device. The input/output interface 53 is connected, for example, to the electrodes 6, an output unit 54 that outputs results of the measurement and other information, or a communication device 55 for transmitting/receiving results of the measurement and other information to/from another device.

The recording medium containing the programs may be a “non-transient, tangible medium” such as a tape, a disc, a card, a semiconductor memory, or programmable logic circuitry.

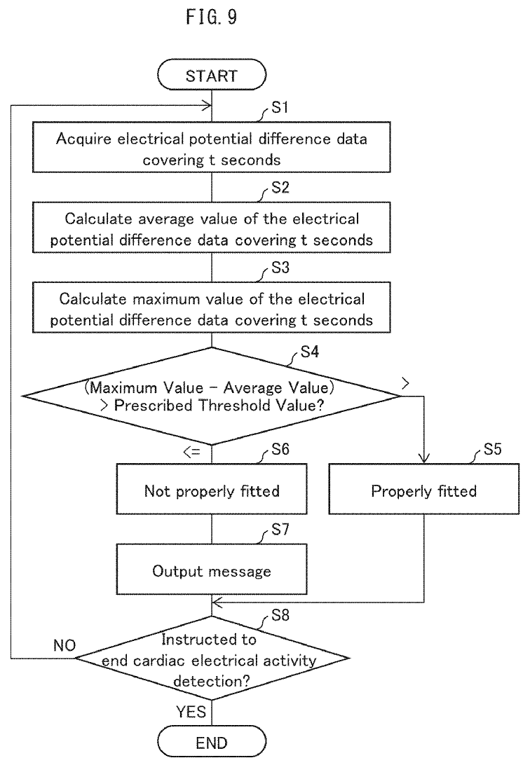

FIG. 9 is a flow chart representing an exemplary flow of a proper fitting check process in the electrocardiograph 4. Referring to FIG. 9, the electrical potential difference detecting unit 42 first acquires electrical potential difference data covering t seconds (e.g., 1 second) (S1). For instance, if the sampling rate is 100 Hz, a total of 100 electrical potential differences is acquired every second. The acquired electrical potential difference data is recorded in the electrical potential difference data recording unit 46.

The proper fitting checking unit 43 calculates an average value of the t-second electrical potential difference data (S2), calculates a maximum value of the data (S3), and then determines if the difference between the maximum and average values is greater than a prescribed threshold value (S4).

If the difference between the maximum and average values is greater than the prescribed threshold value (“>” in S4), the proper fitting checking unit 43 determines that the electrodes 6 are properly fitted (S5). The prescribed threshold value is, for example, 0.1 V.

On the other hand, if the difference between the maximum and average values is less than or equal to the prescribed threshold value (“≤” in S4), the proper fitting checking unit 43 determines that the electrodes 6 are not properly fitted (S6). The check result output unit 44 then alerts the user that the electrodes 6 are not properly fitted (S7). Alternatively, the transmitting unit 45 may transmit the receiving unit 31 information that the electrodes 6 are not properly fitted so that the check result output unit 32 can alert the user of the improper fitting. As a further alternative, both the check result output unit 44 and the check result output unit 32 may alert the user to the improper fitting.

This proper fitting check process is repeated until a cardiac electrical activity detection process is finished (YES in S8). Alternatively, the proper fitting check process may be started and ended when the electrocardiograph 4 is powered on and off respectively.

As an example, the check result output units 44, 32 may alert the user by displaying a message: “Fitting failed” (the alert includes only the fact that the electrodes 6 are not properly fitted) or “Try again” (the alert prompts the user to try to fit the electrodes 6 again). Alternatively, the alert may be an audio output, in which case the audio output is stopped when it is determined that the electrodes 6 have been correctly fitted.

When the proper fitting checking unit 43 determines that the electrodes 6 are properly fitted in the flow chart in FIG. 9, the check result output units 44, 32 may alert the user to the proper fitting. For instance, the check result output units 44, 32 may display a message, “Ready for measurement.” Both or either one of the check result output units 44 and 32 may alert the user to the proper/improper fitting of the electrodes 6.

The proper fitting checking unit 43 may record the results of the check in the electrical potential difference data recording unit 46 after a determination is made in step S4.

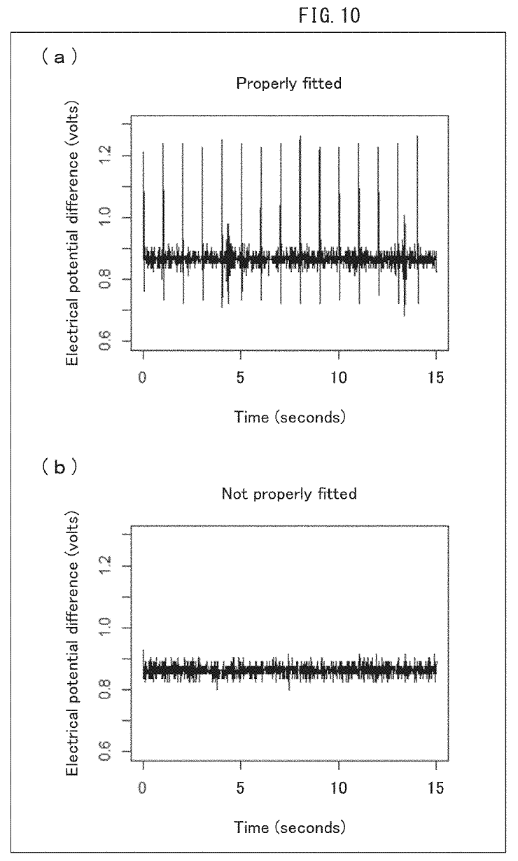

FIG. 10 is a set of diagrams representing exemplary electrocardiographic waveforms, (a) of FIG. 10 representing a waveform obtained when the electrodes 6 are properly fitted and (b) of FIG. 10 representing a waveform obtained when the electrodes 6 are not properly fitted. The waveform in (a) shows substantially regular, distinct peaks, whereas the waveform in (b) shows no such peaks.

In the waveform in (a) of FIG. 10, the difference between the maximum and average values of the electrical potential differences over the 1-second period is approximately 0.36 V. Because the difference between the maximum and average values of the electrical potential differences is greater than or equal to 0.1 V, the proper fitting checking unit 43 determines that the electrodes 6 are properly fitted. On the other hand, in the waveform in (b) of FIG. 10, the difference between the maximum and average values of the electrical potential differences over the 1-second period is approximately 0.06 V. Because the difference between the maximum and average values of the electrical potential differences is less than 0.1 V, the proper fitting checking unit 43 determines that the electrodes 6 are not properly fitted, and the check result output unit 44 alerts the user that the electrodes 6 are not properly fitted.

The proper fitting checking unit 43 does not necessarily check proper fitting by the method described above and may check proper fitting by comparing the maximum value of the electrical potential differences with a prescribed threshold value or by any other method.

The present disclosure is not limited to the description of the embodiments above and may be altered within the scope of the claims. Embodiments based on a proper combination of technical means disclosed in different embodiments are encompassed in the technical scope of the present disclosure. Furthermore, a new technological feature can be created by combining different technological means disclosed in the embodiments.

### CROSS-REFERENCE TO RELATED APPLICATIONS

The present application claims the benefit of priority to Japanese Patent Application, Tokugan, No. 2017-019874, filed on Feb. 6, 2017, the entire contents of which are incorporated herein by reference.

### REFERENCE SIGNS LIST

1, 21 Main Wearable Unit
4 Electrocardiograph (Analysis System)
5 Electrode Belt
6 Electrode
9 Coupling Section
10, 20 Measuring Instrument
11 Length Adjusting Section
13 Extension Belt (Length Adjusting Section)
23 Chest Pad Section
24 Axilla Strap
20 Terminal Device (Analysis System)
43 Proper Fitting Checking Unit (Checking Unit)
100 Electrocardiograph System (Biological Information Measuring System)

1. A measuring instrument comprising:
a main wearable unit to be fitted either to a neck of an animal on which measurement is to be made or close to the neck; and
an electrode belt including an electrode to be fitted, for measurement of biological information, in such a manner that the electrode is in contact with at least either one of left and right axillae of the animal, wherein
the electrode belt has a first end that is connected to the main wearable unit and a second end that is to be connected to the main wearable unit in a location that is closer to a back of the animal than the first end is close to the back of the animal.

2. The measuring instrument according to claim 1, wherein the second end is to be connected to the main wearable unit on the back of the animal.

3. The measuring instrument according to claim 1, wherein the electrode belt includes an elastic portion.

4. The measuring instrument according to claim 1, wherein the electrode belt includes a coupling section to be coupled to the main wearable unit in a detachable manner.

5. The measuring instrument according to claim 1, wherein the electrode belt includes a length adjusting section.

6. The measuring instrument according to claim 1 further comprising:
a chest pad section connected to the main wearable unit and when fitted, positioned in front of a chest of the animal; and
an axilla strap connected to the chest pad section and when fitted, passed under the axilla of the animal.

7. A biological information measuring system comprising:
the measuring instrument according to claim 1; and
an analysis system that analyzes the biological information outputted from the electrode.

8. The biological information measuring system according to claim 7, wherein the analysis system includes a checking unit that analyzes the biological information to determine whether or not the measuring instrument is properly fitted.
